(19) United States
(12) Patent Application Publication
(10) Pub. No.: US 2026/0242255 A1
(43) Pub. Date: Aug. 20, 2026

(54) ANALYSIS DEVICE, ANALYSIS METHOD, ANALYSIS PROGRAM, AND WATER TREATMENT SYSTEM

(71) Applicant: Yokogawa Electric Corporation, Tokyo (JP)

(72) Inventors: Yasuhiro MATSUI, Tokyo (JP); Junji KAMIGUCHI, Tokyo (JP)

(21) Appl. No.: 19/160,496

(22) PCT Filed: Mar. 1, 2024
(86) PCT No.: PCT/JP2024/007884

§ 371 Date: Aug. 28, 2025

(30) Foreign Application Priority Data
Mar. 3, 2023 (JP) 2023-033036

(51) Int. Cl. C02F 1/44; C02F 101/00; C12Q 1/686; C12Q 1/70

(52) U.S. Cl. CPC C02F 1/444; C12Q 1/686; C12Q 1/70; C02F 2101/00; C02F 2209/003

(57) ABSTRACT

A target microorganism is detected to ensure safety of a water quality. Analysis device includes an analyzing unit and a determining unit. The analyzing unit performs PCR analysis with respect to permeate water collected from a membrane that passes water therethrough. The determining unit determines whether the membrane is ruptured based on an analysis result obtained by the analyzing unit. Moreover, performance of the membrane and a sign are grasped.

### TECHNICAL FIELD

The present invention relates to an analysis device, an analysis method, an analysis program, and a water treatment system.

### BACKGROUND ART

A method of detecting a membrane rupture by using air pressure in a conventional water treatment plant (one example of a water treatment system) has been known (for example, refer to Patent Literature 1).

For example, a method of detecting membrane rupture by using air pressure is to detect a membrane rupture by putting water in a first side of a membrane, filling a second side with air, pressurizing the air on the second side, and monitoring the air that leaks to the water side and the pressure at that time.

### CITATION LIST

### Patent Literature

PTL 1: Japanese Laid-open Patent Publication No. 2004-329980

### SUMMARY OF INVENTION

### Technical Problem

The conventional method of detecting membrane rupture has a problem that a minute rupture in a membrane in a water treatment system cannot be detected quickly.

For example, in the method of detecting membrane rupture by using air pressure, it is impossible to detect a membrane rupture with a pore diameter that viruses can pass through, and it is difficult to promptly detect it right after a minute rupture that viruses can pass through occurs.

The present invention has been achieved in view of the above problems, and it is an object of the present invention to detect a minute rupture in a membrane in a water treatment system quickly.

### Solution to Problem

According to one aspect of embodiments, an analysis device includes: an analyzing unit configured to perform PCR analysis with respect to permeate water collected from a membrane that passes water therethrough; and a determining unit configured to determine whether the membrane is ruptured based on an analysis result obtained by the analyzing unit.

### Advantageous Effects of Invention

According to one embodiment, a minute rupture in a membrane in a water treatment system can be detected quickly.

### BRIEF DESCRIPTION OF DRAWINGS

FIG. 1 is a diagram illustrating a configuration example of a water treatment system according to an embodiment.

FIG. 2 is a diagram illustrating a configuration example of a UF membrane device.

FIG. 3 is a diagram illustrating a configuration example of the UF membrane device and peripheral devices.

FIG. 4 is a diagram illustrating an arrangement example of UF membranes and analysis devices.

FIG. 5 is a diagram illustrating a configuration example of an RO membrane device.

FIG. 6 is a diagram illustrating an arrangement example of RO membranes and the analysis devices.

FIG. 7 is a diagram illustrating a configuration example of the analysis device.

FIG. 8 is a flowchart illustrating a flow of processing of the analysis device.

FIG. 9 is a diagram explaining a hardware configuration example.

### DESCRIPTION OF EMBODIMENTS

Hereinafter, embodiments of an analysis device, an analysis method, an analysis program, and a water treatment system disclosed in the present application will be explained in detail based on the drawings. The embodiments explained herein are not intended to limit the present invention. Moreover, identical reference symbols are assigned to identical components, and duplicated explanation is omitted as appropriate. Furthermore, the respective embodiments can be combined appropriately within a range not causing a contradiction.

Water treatment of sewage, and drainage of rainwater and the like is generally categorized into three main processes including primary treatment, secondary treatment, and tertiary treatment.

In the primary treatment, removal of large solid materials such as foreign objects included in the drainage is performed. In the secondary treatment, organic matter that could not be eliminated in the primary treatment is removed by using microorganisms (bacteria). In the secondary treatment, for example, activated sludge treatment, nitrification-denitrification treatment, and the like are performed. In the tertiary treatment, removal of suspended solids that could not be eliminated in the secondary treatment by sediment removal is performed. In the tertiary treatment, removal of suspended solids by sand filtration or membrane filtration is performed.

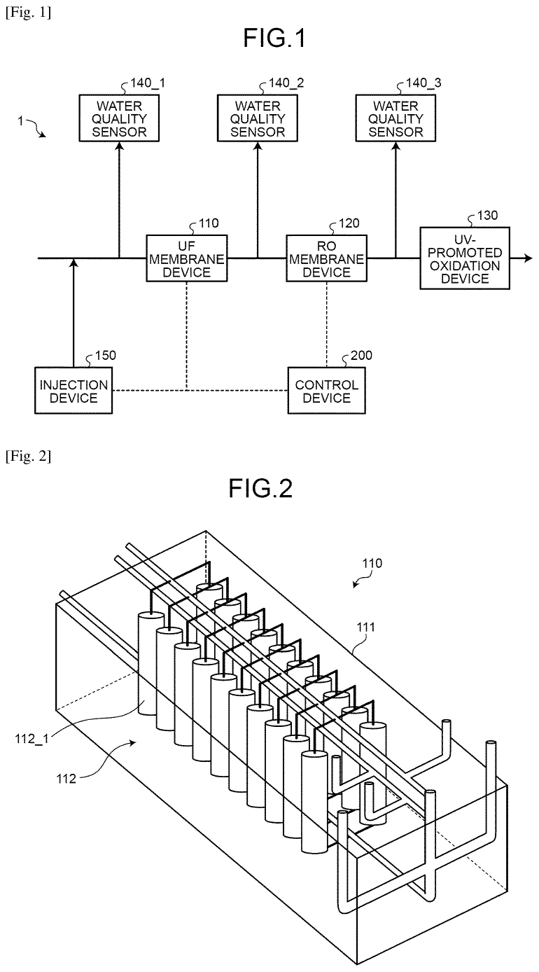

FIG. 1 is a diagram illustrating a configuration of a water treatment system according to an embodiment. A water treatment system 1 illustrated in FIG. 1 includes a ultra-filtration membrane (UF membrane) device 110, a reverse osmosis membrane (RO membrane) device 120, a UV-promoted oxidation device 130, a water quality sensor 140_1, a water quality sensor 140_2, a water quality sensor 140_3, an injection device 150, and a control device 200. Note that the water treatment system illustrated in FIG. 1 is one example, and the water treatment system according to the present embodiment may have components other than the components illustrated in FIG. 1.

The water treatment system 1 recycles wastewater including sewage and rainwater into domestic water and drinking water. To the water treatment system 1, for example, treated water obtained by subjecting wastewater to activated sludge treatment, nitrification-denitrification treatment, and the like (that is, treated water that has undergone the primary treatment and the secondary treatment) is supplied. The water treatment system 1 subjects the supplied treated water to membrane treatment and the like. That is, the water treatment system 1 mainly performs the tertiary treatment described above.

The treated water treated by the water treatment system 1 is disinfected with, for example, chlorine or the like, and is used as domestic water and drinking water.

The activated sludge treatment, the nitrification-denitrification treatment, and the like are performed, for example, in sewage treatment facility. The water treatment system 1 subjects, for example, drainage treated at sewage treatment facility to membrane treatment and the like.

Supply water that is supplied to the UF membrane device 110 is also denoted as membrane-filtered supply water. Supply water that is supplied to the RO membrane device 120 is denoted as reverse-osmosis-membrane supply water. Supply water that is supplied to the UV-promoted oxidation device 130 is also denoted as UV supply water.

### UF Membrane Device 110

UF membrane device 110 removes microorganisms and particulate matter from the supply water with a filtration membrane. The UF membrane device 110 includes multiple UF membranes. The UF membrane device 110 subjects the UF-membrane supply water to membrane filtration using the UF membranes, to remove microorganisms and particulate matter. The UF membrane device 110 supplies membrane-filtered permeate water (UF membrane permeate water) after membrane filtration to the RO membrane device 120.

A configuration of the UF membrane device 110 will be explained by using FIG. 2. FIG. 2 is a diagram illustrating a configuration example of the UF-membrane device.

As illustrated in FIG. 2, the UF membrane device 110 includes a container 111 that houses a UF membrane module (UF membrane element group) 112 thereinside. The UF membrane module 112 includes multiple UF membranes (UF membrane elements).

In FIG. 2, reference symbols are omitted except for one UF membrane 112_1. Shapes congruent to a shape (cylinder) indicating the UF membrane 112_1 in FIG. 2 all express UF membranes.

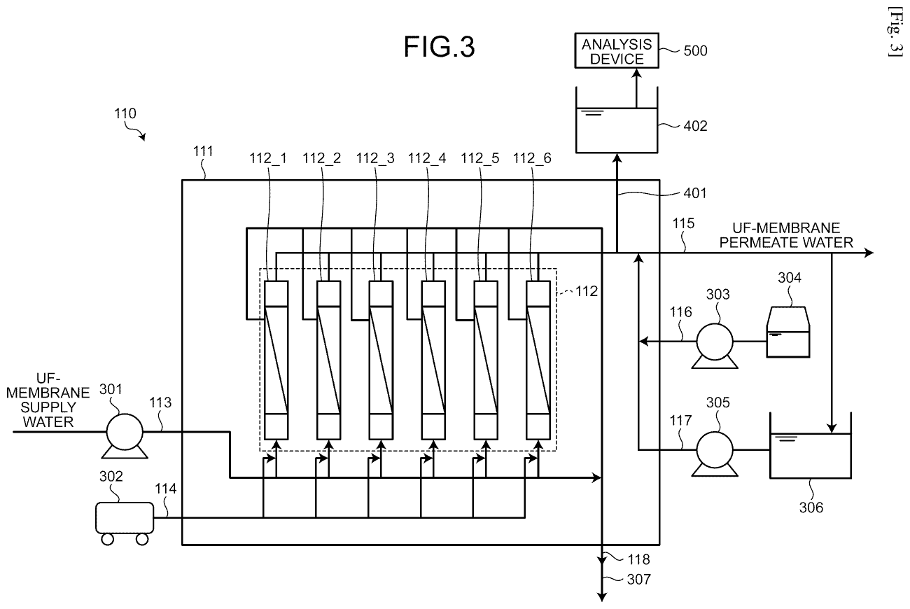

The configuration of the UF membrane device 110 will be explained in more detail by using FIG. 3. FIG. 3 is a diagram illustrating a configuration example of the UF membrane device and peripheral devices.

As illustrated in FIG. 3, the UF membrane module 112 housed in the container 111 includes the UF membrane 112_1, a UF membrane 112_2, a UF membrane 112_3, and a UF membrane 112_4. The number of UF membranes included in the container 111 is not limited to the one illustrated.

The container 111 includes an inlet pipe 113 to supply the UF-membrane supply water to the UF membrane module 112. The inlet pipe 113 is connected to a pump 301. The pump in the present embodiment is a feeder pump to supply the supply water to the UF membranes and the like.

The container 111 includes an inlet pipe 114 to supply compressed air for air cleaning to the UF membrane module 112. The inlet pipe 114 is connected to an air compressor 302 that supplies compressed air.

The container 111 includes a permeate pipe 115 for collecting water subjected to membrane filtration from the UF membrane module 112. The permeate pipe 115 supplies water after membrane filtration to a subsequent treatment device (for example, the RO membrane device 120).

Filtration membranes including the UF membranes are periodically cleaned to remove blockage. For example, as cleaning of UF membranes, there are backwashing (cleaning using filtered water), cleaning using chemicals (for example, sulfuric acid, citric acid, and sodium hypochlorite) (maintenance cleaning (MC)), and chemical cleaning in which high-concentration chemicals are used and prolonged immersion is performed (recovery cleaning (RC), and the like.

The container 111 includes an inlet pipe 116 to supply chemicals for chemical cleaning to the UF membrane module 112. The inlet pipe 116 is connected to a chemical tank 304 in which the chemical for chemical cleaning is stored through a pump 303.

The container 111 includes an inlet pipe 117 to supply water for backwashing to the UF membrane module 112. the inlet pipe 117 is connected to a backwashing tank 306 that stores water for backwashing through a pump 305.

The container 111 includes a drainpipe 118 to convey wastewater from the UF membrane module 112. The drainpipe 118 is connected to a drainpipe 307 outside the container 111.

A permeate pipe 401 passes a portion of the UF membrane permeate water as a sample to a tank 402. The sample acquired by the permeate pipe 401 is used for detection of membrane rupture. A detection method of membrane rupture will be described later.

Moreover, the container 111 has a connecting pipe to connect the container 111 with other devices. The connecting pipe connects a device that performs the secondary treatment or the RO membrane device 120 with the container 111. Moreover, the pipes including the inlet pipe 113 and the permeate pipe 115 explained so far may serve as the connecting pipe.

The connecting pipe is a mechanism to make the container 111 detachable with respect to the water treatment system 1. The connecting pipe has a fitting for low pressure. The fitting is, for example, a screw-in fitting, a flange, or the like made of stainless steel or polyvinyl chloride material. The connecting pipe is only required to be able to withstand pressure of, for example, approximately 0.49 MPa or lower.

An analysis device 500 detects a rupture in the UF membrane included in the UF membrane module 112 based on the sample of permeate water stored in the tank 402.

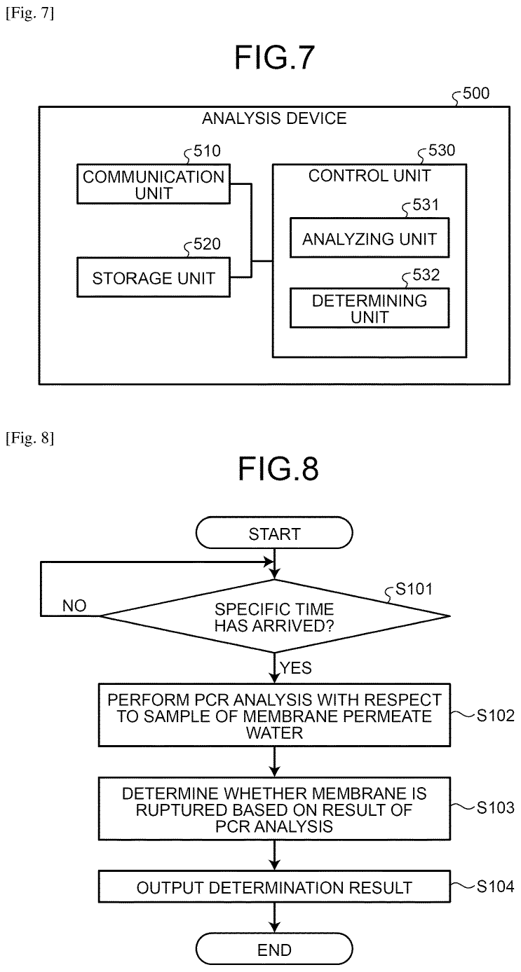

A configuration of the analysis device 500 will be explained by using FIG. 7. FIG. 7 is a diagram illustrating a configuration example of the analysis device. As illustrated in FIG. 7, the analysis device 500 includes a communication unit 510, a storage unit 520, and a control unit 530. Functional units included in the analysis device 500 are not limited to the ones illustrated, and a functional unit such as an interface that transmits and receives data with an output device, such as a display and a speaker may be included.

The communication unit 510 is a processing unit that controls communication with other devices, and is implemented by, for example, a communication interface or the like.

The storage unit 520 is a processing unit that stores various kinds of data, various kinds of programs that are executed by the control unit 530, and the like. The storage unit 520 is implemented by, for example, a memory, a hard disk, and the like. The storage unit 520 stores various kinds of data that is generated by processing performed by the analysis device 500, such as data acquired during various kinds of processing performed by the control unit 530 and processing results acquired as a result of performing various kinds of processing.

The control unit 530 is a processing unit that controls the entire analysis device 500. The control unit 530 is implemented by, for example, a processor or the like. The control unit 530 includes an analyzing unit 531 and a determining unit 532.

The analyzing unit 531 performs polymerase chain reaction (PCR) analysis with respect to permeate water acquired from a membrane that permeates water. The genetic nucleic acids of microorganisms present in the permeate water is amplified using the PCR method. Furthermore, reformation (hybridization) of amplified nucleic acids and fluorescent nucleic acid probes is performed. The analyzing unit 531 then identifies a kind of microorganism present in the permeate water by detecting emission of light. The analyzing unit 531 may be replaced with a conventional PCR device that performs a series of processing relating to PCR explained herein. In this case, the determining unit 532 acquires a result of PCR analysis performed by the PCR device.

The determining unit 532 determines whether a membrane is ruptured based on the analysis result by the analyzing unit 531. The determining unit 532 determines that a rupture of a size according to the kind of the microorganism has occurred when it is found that a microorganism selected in advance is present in the permeate water.

For example, when a microorganism included in the supply water to the UF membrane is also detected in the UF membrane by the PCR analysis, it is considered that the microorganism has passed through the UF membrane. Furthermore, when the detected microorganism is larger than a pore diameter of the UF membrane, it is suspected that the organism has passed through a ruptured area. By using this, the analysis device 500 performs detection of membrane rupture.

Microorganisms include protozoa, bacteria, viruses, and the like. Protozoa include, for example, Cryptosporidium and Giardia. Bacteria include Escherichia coli, Staphylococcus, Vibrio cholerae, Mycobacterium tuberculosis, Helicobacter pylori, and the like. Viruses include Norovirus, Adenovirus, enteric viruses, Pepper Mild Mottle Virus (PMMoV), and the like.

The analyzing unit 531 detects presence of target microorganism among microorganisms including viruses in the permeate water by the PCR analysis. Thus, the analysis device 500 can achieve not only rupture detection but also ensuring safety of water quality in membrane filtration (MF membrane, UF membrane, RO membrane).

The determining unit 532 determines not only whether a membrane is ruptured, but further determines whether there is a sign of rupture in the membrane, and whether the membrane is high-performance.

For example, the determining unit 532 determines that the membrane is high-performance if a ratio of the number of times that the membrane is determined to be ruptured to the total number of times that determination of membrane rupture is performed is equal to or lower than a threshold.

Moreover, even when presence of a pre-selected microorganism is not identified by the analyzing unit 531, the determining unit 532 determines that there is a sign of membrane rupture when presence of a specific microorganism further smaller than the microorganism is identified.

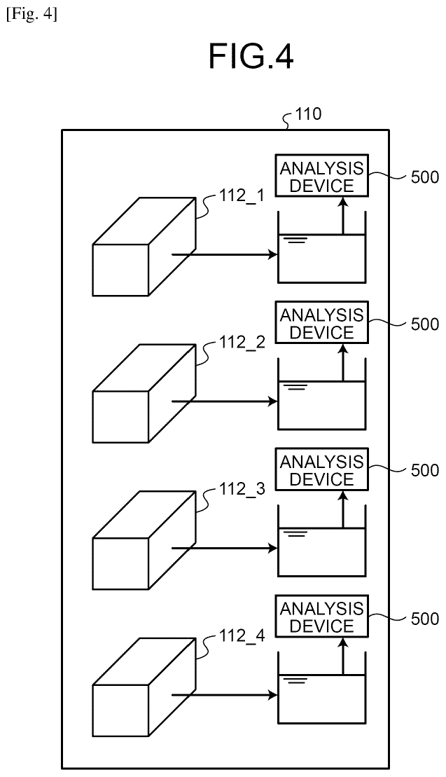

FIG. 4 is a diagram illustrating an arrangement example of the UF membranes and the analysis devices. The analysis device 500 is arranged for each of the UF membranes (the UF membrane 112_1, the UF membrane 112_2, the UF membrane 112_3, the UF membrane 112_4) included in the UF membrane device 110. Alternatively, it may be configured such that a single unit of the analysis device 500 performs detection of membrane rupture for the multiple UF membranes. Furthermore, a function equivalent to the analysis device 500 may be provided in the control device 200.

### RO Membrane Device 120

Returning back to FIG. 1, to the RO membrane device 120, treated water by the UF membrane device 110 is supplied. The RO membrane device 120 removes impurities, such as ions and salts from the supply water. The RO membrane device 120 includes an RO membrane.

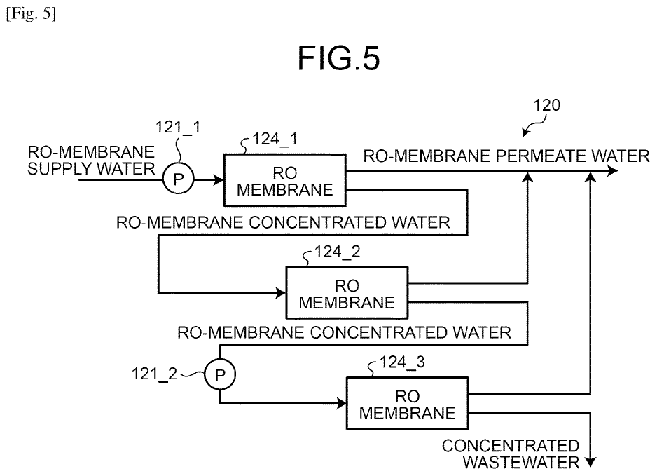

FIG. 5 is a diagram illustrating a configuration example of the RO membrane device. The RO membrane device 120 illustrated in FIG. 5 includes a pump 121_1 and a pump 121_2. Moreover, the UF membrane device 110 includes an RO membrane 124_1, an RO membrane 124_2, and an RO membrane 124_3.

To the RO membrane 124_1, the RO-membrane supply water is supplied by using the pump 121_1. The RO membrane 124_1 separates the RO-membrane supply water into RO-membrane permeate water (RO-membrane filtered water) and RO-membrane concentrated water. The RO membrane 124_1 supplies the RO-membrane concentrated water to the RO membrane 124_2.

The RO membrane 124_2 separates the RO-membrane concentrated water into RO-membrane permeate water and RO-membrane concentrated water. The RO membrane 124_2 supplies the RO-membrane concentrated water to the RO membrane 124_3.

To the RO membrane 124_3, the RO-membrane concentrated water is supplied from the RO membrane 124_2 by using the pump 121_2. The RO membrane 124_3 separates the RO concentrated water into RO-membrane permeate water and concentrated wastewater by using the RO membrane. The RO membrane 124_3 drains the concentrated wastewater to the outside of the RO membrane device 120.

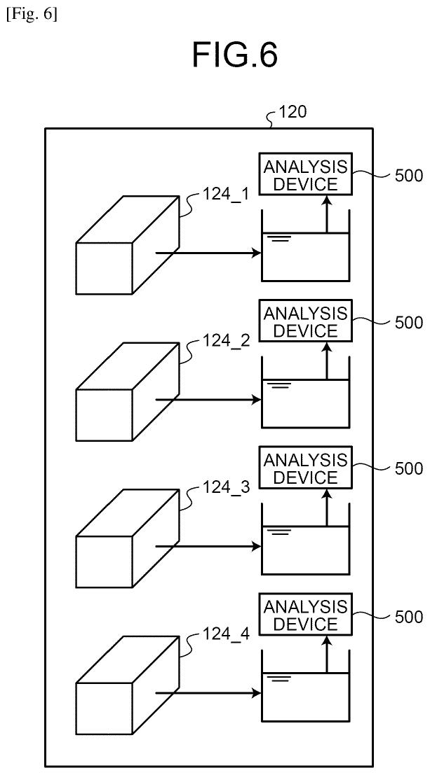

The target for membrane rupture detection is not limited to UF membranes, and it may be RO membranes, and the like. FIG. 6 is a diagram illustrating an arrangement example of the RO membranes and the analysis devices. The analysis device 500 is arranged for each of the RO membranes (the RO membrane 124_1, the RO membrane 124_2, the RO membrane 124_3, and an RO membrane 124_4) included in the RO membrane device 120. Alternatively, it may be configured such that a single unit of the analysis device 500 perform membrane rupture detection for the multiple RO membranes.

Returning back to FIG. 1, the RO membrane device 120 supplies RO membrane permeate water to the UV-promoted oxidation device 130.

### UV-Promoted Oxidation Device 130

The UV-promoted oxidation device 130 performs UV advanced oxidation process (AOP) (promoted oxidation using ultraviolet rays) with respect to the UV supply water. Thus, the UV-promoted oxidation device 130 oxidizes and decomposes trace chemical substances (for example, NDMA) contained in the UV supply water.

### Water Quality Sensor 140

The water quality sensor 140 measures a water quality of supply water of respective parts of the water treatment system 1, filtered water (permeate water), and the like. In the example of FIG. 1, the water treatment system 1 includes a water quality sensor 140_1, a water quality sensor 140_2, and 140_3.

The water quality sensor 140_1 measures a water quality at an inlet portion of the UF membrane device 110. The water quality sensor 140_1 measures a water quality of the UF-membrane supply water.

The water quality sensor 140_1 measures, for example, at least one of water temperature, pH value, oxidation-reduction potential (ORP), ammonia nitrogen content, nitrogen compound content, turbidity, ultraviolet absorbance, electrical conductivity, and total organic carbon (TOC) value of filtered supply water.

The water quality sensor 140_2 measures a water quality at an inlet portion of the RO membrane device 120 (or an outlet portion of the UF membrane device 110). The water quality sensor 140_2 measures a water quality of the RO-membrane supply water (or the UF-membrane permeate water).

The water quality sensor 140_3 measures a water quality of an inlet portion of the UF-promoted oxidation device 130 (or an outlet portion of the RO-membrane device 120). The water quality sensor 140_3 measures a water quality of the RO-membrane permeate water.

The water quality sensor 140 included in the water treatment system 1 is not limited to the example in FIG. 1. For example, the water treatment system 1 may include the water quality sensor 140 that is not illustrated in FIG. 1, such as the water quality sensor 140 that measures a water quality of the UV supply water, may also be included.

The water quality sensor 140_1 outputs a measurement result to the control device 200.

The injection device 150 injects a chemical solution into the inlet portion of the UF membrane device 110 in accordance with an instruction from the control device 200. The injection device 150 injects, for example, sodium hypochlorite to the inlet portion of the UF membrane device 110. Moreover, the injection device 150 injects, for example, chemical solutions, such as ammonium sulfate and ammonium chloride, other than sodium hypochlorite to the inlet portion of the UF membrane device 110.

The control device 200 controls respective components of the water treatment system 1. The control device 200 may perform control in accordance with a request from the analysis device 500.

An example of processing of the analyzing unit 531 and the determining unit 532 will be explained by using FIG. 8. FIG. 8 is a flowchart illustrating a flow of the processing of the analyzing unit.

The processing in FIG. 8 will be explained. As illustrated in FIG. 8, first, the analyzing unit 531 waits until a specific time arrives (step S101: NO). When the specific time arrives (step S101: YES), the analyzing unit 531 performs the PCR analysis with respect to a sample of membrane permeate water (step S102).

For example, the sample of membrane permeate water is collected from the UF membrane module 112, and is stored in the tank 402. The tank 402 can store a maximum of one day's worth of permeate water.

At step S101, the analyzing unit 531 may wait until specific time passes. That is, the analyzing unit 531 performs processing at S102 and later in regular time intervals.

The determining unit 532 determines whether the membrane is ruptured based on a result of the PCR analysis (step S103). Furthermore, the analysis device 500 outputs a determination result through the communication unit 510 (corresponding to an output unit) (step S104). For example, the communication unit 510 transmits the determination result to a terminal device used by an operator of the water treatment system 1, or the like.

The time interval for performing processing at step S102 and later may be within a range of, for example, from 1 hour to 24 hours. In that case, detection of a membrane rupture can be performed at least once a day. Moreover, for example, the membrane rupture may be performed, for example, at intervals of 2.5 hours.

California State Water Resources Control Board defines microbiological measurement conducted within a time range of 3 to 18 hours as rapid measurement. The analyzing unit 531 can acquire an analysis result within 24 hours at the latest by performing the PCR analysis using the known rapid measurement. In that case, if processing of detecting a membrane rupture is started when the operator of the water treatment system 1 starts work at 8 o'clock every morning, the operator can review the detection result in the afternoon on the same day or the next day, and can take necessary actions as needed.

At step S103, the determining unit 532 checks whether a nucleic acid originated from a target microorganism is present in the permeate water based on the result of the PCR analysis. The determining unit 532 determines that the membrane is ruptured when the result of the PCR analysis shows that a nucleic acid originated from the target microorganism is present in the permeate water. That is, the determining unit 532 determines that a rupture of a size according to the size of the target microorganism detected by the PCR analysis by the analyzing unit 531 has occurred.

The target microorganism is selected in advance according to the size of a rupture desired to be detected, and the pore diameter of a UF membrane. For example, the target microorganism is determined according to the pore diameter and the pore diameter distribution (for example, 0.02 μm to 0.1 μm) of the UF membrane, the shape and the size of the microorganism (for example, 20 nm or larger diameter in the case of a virus), and membrane adsorption characteristics.

Furthermore, at step S103, the analyzing unit 531 may perform the PCR analysis with respect to permeate water over a certain period (for example, 1 day). In this case, at step S103, the determining unit 532 determines whether a rupture has occurred in the period according to the sensitivity characteristics of the PCR analysis and the result of the PCR analysis.

Thus, for example, using a highly sensitive PCR and the sensitivity, it is possible to determine whether a membrane rupture has occurred within the time by storing the permeate water over an extended period of time, and checking presence of the target organism stored during the same storage time.

As a result, a minute membrane rupture that has conventionally been impossible to be detected can be detected, and the water quality assurance and the membrane quality assurance (integrity (no ruptures)) can be highly achieved.

The analyzing unit 531 may perform the PCR analysis with respect to permeate water concentrated after collection. Thus, the PCR analysis can be performed with higher accuracy and, as a result, the detection accuracy of membrane rupture is improved.

As one example, the water treatment system 1 performs concentration of permeate water by the following procedure, and stores the concentrated permeate water in the tank 402 as a sample. The analysis device 500 performs membrane rupture detection with respect to the concentrated permeate water.

That is, the water treatment system 1 mixes permeate water and a solution (for example, magnesium chloride solution), to obtain a mixture solution. Next, the water treatment system 1 supplies the mixture solution to a negatively charged membrane. At the negatively charged membrane, positive ions and viruses in the mixture solution are captured. Moreover, the water treatment system 1 discharges liquid after the positive ions and the viruses are removed at the negatively charged membrane.

Subsequently, the water treatment system 1 performs acid cleaning of the negatively charged membrane with an acidic solution (for example, sulfuric acid solution). Thus, the positive ions are removed from the negatively charged membrane.

The water treatment system 1 removes the viruses captured by the negatively charged membrane by flowing an alkaline solution (for example, sodium hydroxide solution) through the negatively charged membrane. Thus, the water treatment system 1 can acquire a solution with viruses concentrated with the alkaline solution.

At step S104, the analysis device 500 may output information relating to a kind of the microorganism detected by the analyzing unit 531 and information relating to the size of a rupture according to the kind of the organism.

For example, the analysis device 500 outputs information indicating that a membrane rupture has been detected and the size of the microorganism detected by the PCR analysis in set. Thus, the operator of the water treatment system 1 can easily determine whether emergency responses to the membrane rupture is necessary, and the like.

Moreover, as described previously, the target of detection of membrane rupture by the analysis device 500 is not limited to UF membranes and RO membranes. For example, by performing the PCR analysis with respect to permeate water acquired from micro filtration membranes (MF membranes) or nano filtration membranes (NF membrane), the analysis device 500 may detect membrane rupture of these membranes.

Furthermore, because the pore diameter of the MF membrane and the pore diameter distribution is from 1 μm to 10 μm, when a membrane rupture of the MF membrane is to be detected, bacteria larger than viruses (diameter of 1 μm to 10 μm) and protozoa (3 μm or larger) may be selected as the target microorganism.

For the RO membrane and the NF membrane, the pore diameter of the membrane cannot be defined. Therefore, as for the RO membrane and the NF membrane, a molecular weight of ions that can be excluded (fractionated) in a molecular level (generally, 300 Da, Da represents the molecular mass of a molecule) is defined as the removal capacity. The RO membrane and the NF membrane have separation capability to completely block microorganisms larger in diameter than viruses.

On the other hand, because the RO membrane and the NF membrane are elementalized, physical and mechanical parts, such as a sealing portion (brine seal), a fusion portion of membranes, a water stopper gasket that separates suppl water, permeate water, and concentrated water, and an O-ring, are included. Moreover, microorganisms can flow into permeate water due to a rupture of such a part. Therefore, according to the analysis device 500, it is possible to detect a rupture of not only a membrane, but also another part.

### Effects of Embodiments

The analysis device 500 includes the analyzing unit 531 and the determining unit 532. The analyzing unit 531 performs the PCR analysis with respect to permeate water acquired from a membrane that let water pass therethrough. The determining unit 532 determines whether the membrane is ruptured, whether there is a sign of rupture in the membrane, and whether the membrane is high-performance based on an analysis result by the analyzing unit 531.

According to the present embodiment, a minute rupture of a membrane in the water treatment system 1 can be detected quickly without taking a process of applying pressure with air.

### System

The processing procedure, the control procedure, specific names and information including various kinds of data and parameters described in the above document and the drawings can be changed arbitrarily unless otherwise specified.

Moreover, the respective components of the respective devices illustrated are of functional concept, and it is not necessarily required to be configured physically as illustrated. That is, specific forms of distribution and integration of the respective devices are not limited to the ones illustrated, and all or some thereof can be configured to be distributed or integrated functionally or physically in arbitrary units according to various kinds of loads, usage conditions, and the like.

Furthermore, as for the respective processing functions performed by the respective devices, all or an arbitrary part thereof can be implemented by a CPU and a computer program that is analyzed and executed by the CPU, or can be implemented as hardware by wired logic.

### Hardware

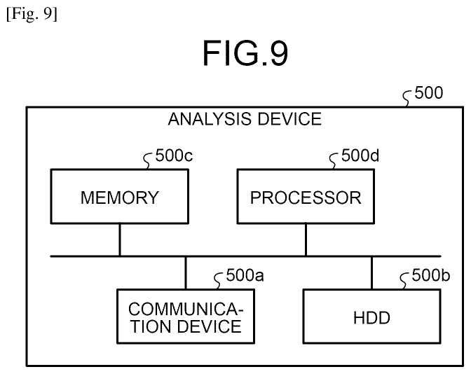

Next, a hardware configuration of the analysis device 500 will be explained. FIG. 9 is a diagram explaining a hardware configuration example. As illustrated in FIG. 9, the analysis device 500 includes a communication device 500a, a hard disk drive (HDD) 500b, a memory 500c, and a processor 500d. Moreover, the respective components illustrated in FIG. 9 are connected to one another through a bus or the like.

The communication device 500a is a network interface card or the like, and performs communication with other servers. The HDD 500b stores a program to operate the functions illustrated in FIG. 7 and a DB.

The processor 500d reads the program that implements processing similar to that of the respective processing units illustrated in FIG. 7 from the HDD 500b or the like and expands it to the memory 500c, to thereby operate processes to implement the respective functions explained in FIG. 7 and the like. For example, this process performs functions similar to those of the respective processing units included in the analysis device 500. Specifically, the processor 500d reads out a program having functions similar to those of the analyzing unit 531, the determining unit 532, and the like from the HDD 500b or the like. The processor 500d performs the process to perform the processing similar to that of the analyzing unit 531 and the determining unit 532.

As described, the analysis device 500 operates as a device that performs a analysis method by reading a program. Moreover, the analysis device 500 can implement functions similar to the embodiment described above by reading the program described above from a recording medium by a medium reader device, and by executing the read program described above also. Programs in other embodiments are not limited to be executed by the analysis device 500. For example, the present invention can be applied similarly also to a case in which the program is executed by other computers or servers, or a case in which the program is executed by these in cooperation.

This program can be distributed through a network such as the Internet. Furthermore, this program can be recorded on a computer-readable recording medium, such as a hard disk, a flexible disk (FD), a CD-ROM, a magneto optical disk (MO), and a digital versatile disk (DVD), and can be executed by being read by a computer from the recording medium.

### Others

Some examples of combinations of the disclosed technical features are described in the following.
(1)

An analysis device comprising:
an analyzing unit configured to perform PCR analysis with respect to permeate water collected from a membrane that passes water therethrough; and
a determining unit configured to determine whether the membrane is ruptured based on an analysis result obtained by the analyzing unit.
(2)

The analysis device according to (1), wherein
the determining unit is configured to further determine whether there is a sign of rupture of the membrane, and whether the membrane is high-performance based on an analysis result obtained by the analyzing unit.
(3)

The analysis device according to (1) or (2), wherein
the analyzing unit is configured to detect presence of a target microorganism out of microorganisms including a virus in the permeate water, by the PCR analysis.
(4)

The analysis device according to any one of (1) to (3), wherein
the analyzing unit is configured to perform PCR analysis with respect to the permeate water over a certain period of time, and
the determining unit is configured to determine whether a rupture has occurred in the period of time according to sensitivity characteristics of the PCR analysis and a result of the PCR analysis.
(5)

The analysis device according to any one of (1) to (4), wherein
the determining unit is configured to determine that a rupture in a size according to a size of a microorganism has occurred in the membrane, the microorganism detected by the PCR analysis performed by the analyzing unit.
(6)

The analysis device according to any one of (1) to (5), wherein
the analyzing unit is configured to perform PCR analysis with respect to permeate water collected from a UF membrane.
(7)

The analysis device according to any one of (1) to (6), wherein
the analyzing unit is configured to perform PCR analysis with respect to permeate water collected from an MF membrane.
(8)

The analysis device according to any one of (1) to (7), wherein
the analyzing unit is configured to perform PCR analysis with respect to permeate water that is concentrated after collection.
(9)

The analysis device according to any one of (1) to (8), further comprising
an output unit configured to output information relating to a kind of microorganism detected by the analyzing unit, and information relating to a size of a rupture according to the kind of the microorganism.
(10)

An analysis method that is performed by a computer, the method comprising:
a analyzing process of performing PCR analysis with respect to permeate water collected from a membrane that passes water therethrough; and
a determining process of determining whether the membrane is ruptured based on an analysis result obtained by the analyzing process.
(11)

The analysis method according to (10), wherein
the determining process further includes determining whether there is a sign of rupture of the membrane, and whether the membrane is high-performance based on an analysis result obtained by the analyzing process.
(12)

The analysis method according to (10) or (11), wherein
the analyzing process includes detecting presence of a target microorganism out of microorganisms including viruses in the permeate water by the PCR analysis.
(13)

The analysis method according to any one of (10) to (12), wherein
the analyzing process includes performing PCR analysis with respect to the permeate water over a certain period of time, and
the determining process includes determining whether a rupture has occurred in the period of time according to sensitivity characteristics of the PCR analysis and a result of the PCR analysis.
(14)

The analysis method according to any one of (10) to (13), wherein
the determining process includes determining that a rupture in a size according to a size of a microorganism has occurred in the membrane, the microorganism detected by the PCR analysis at the analyzing process.
(15)

The analysis method according to any one of (10) to (14), wherein
the analyzing process includes performing PCR analysis with respect to permeate water that is collected from a UF membrane.
(16)

The analysis method according to any one of (10) to (15), wherein
the analyzing process includes performing PCR analysis with respect to permeate water that is collected from an MF membrane.
(17)

The analysis method according to any one of (10) to (16), wherein
the analyzing process includes performing PCR analysis with respect to permeate water that is concentrated after collection.
(18)

The analysis method according to any one of (10) to (17), further including
an outputting process of outputting information relating to a kind of an organism detected by the analyzing process, and information relating to a size of a rupture according to the kind of the microorganism.
(19)

An analysis program that causes a computer to execute:
an analyzing step of performing PCR analysis with respect to permeate water collected from a membrane that passes water therethrough; and
a determining step of determining whether the membrane is ruptured based on an analysis result obtained by the analyzing step.
(20)

The analysis program according to (19), wherein
the determining step further includes determining whether there is a sign of rupture of the membrane, and whether the membrane is high-performance based on an analysis result obtained by the analyzing step.
(21)

The analysis program according to (19) or (20), wherein
the analyzing step includes detecting presence of a target microorganism out of microorganisms including viruses in the permeate water by the PCR analysis.
(22)

The analysis program according to any one of (19) to (21), wherein
the analyzing step includes performing PCR analysis with respect to the permeate water over a certain period of time, and
the determining step includes determining whether a rupture has occurred in the period of time according to sensitivity characteristics of the PCR analysis and a result of the PCR analysis.
(23)

The analysis program according to any one of (19) to (22), wherein
the determining step includes determining that a rupture in a size according to a size of a microorganism has occurred in the membrane, the microorganism detected by the PCR analysis at the analyzing step.
(24)

The analysis program according to any one of (19) to (23), wherein
the analyzing step includes performing PCR analysis with respect to permeate water that is collected from a UF membrane.
(25)

The analysis program according to any one of (19) to (24), wherein
the analyzing step includes performing PCR analysis with respect to permeate water that is collected from an MF membrane.
(26)

The analysis program according to any one of (19) to (25), wherein
the analyzing step includes performing PCR analysis with respect to permeate water that is concentrated after collection.
(27)

The analysis program according to any one of (19) to (26), further including
an outputting step of outputting information relating to a kind of an organism detected by the analyzing step, and information relating to a size of a rupture according to the kind of the microorganism.
(28)

A water treatment system comprising:
a container; and
an analysis device, wherein
the container includes
a membrane module including a plurality of membranes that pass water therethrough;
a pipe to supply water to the membrane module;
a pipe to collect water from the membrane module; and
a pipe to connect to a device included in a system that treats sewage, and
the analysis device includes
an analyzing unit configured to perform PCR analysis with respect to permeate water collected from at least either one of the membranes;
a determining unit configured to determine whether the membrane is ruptured based on an analysis result obtained by the analyzing unit; and
an output unit configured to output a determination result obtained by the determining unit.
(29)

The water treatment system according to (28), wherein
the determining unit further determines whether there is a sign of rupture of the membrane, and whether the membrane is high-performance based on an analysis result obtained by the analyzing unit.
(30)

The water treatment system according to (28) or (29), wherein
the analyzing unit detects presence of a target microorganism out of microorganisms including viruses in the permeate water by the PCR analysis.
(31)

The water treatment system according to any one of (28) to (30), wherein
the analyzing unit performs PCR analysis with respect to the permeate water over a certain period of time, and
the determining unit determines whether a rupture has occurred in the period of time according to sensitivity characteristics of the PCR analysis and a result of the PCR analysis.
(32)

The water treatment system according to any one of (28) to (31), wherein
the determining unit determines that a rupture in a size according to a size of a microorganism has occurred in the membrane, the microorganism detected by the PCR analysis by the analyzing unit.
(33)

The water treatment system according to any one of (28) to (32), wherein
the analyzing unit performs PCR analysis with respect to permeate water that is collected from a UF membrane.
(34)

The water treatment system according to any one of (28) to (33), wherein
the analyzing unit performs PCR analysis with respect to permeate water that is collected from an MF membrane.
(35)

The water treatment system according to any one of (28) to (34), wherein
the analyzing unit performs PCR analysis with respect to permeate water that is concentrated after collection.
(36)

The water treatment system according to any one of (28) to (35), further including
an output unit that outputs information relating to a kind of an organism detected by the analyzing unit, and information relating to a size of a rupture according to the kind of the microorganism.

### Reference Signs List

1 Water treatment system
110 UF membrane device
111 Container
112 UF membrane module
112_1, 112_2, 112_3, 112_4 UF membrane
113, 114, 116, 117 Inlet pipe
115, 401 Permeate pipe
118, 307 Drainpipe
120 RO membrane device
121_1, 121_2, 301, 303, 305 Pump
124_1, 124_2, 124_3 RO membrane
130 UV-promoted oxidation device
140_1, 140_2, 140_3 Water quality sensor
150 Injection device
200 Control device
302 Air compressor
304 Chemical tank
306 Backwashing tank
402 Tank
500 Analysis device
510 Communication unit
520 Storage unit
530 Control unit
531 Analyzing unit
532 Determining unit

1. An analysis device comprising:
a processor configured to:
perform PCR analysis with respect to permeate water collected from a membrane that passes water therethrough; and
determine whether the membrane is ruptured based on an analysis result obtained

2. The analysis device according to claim 1, wherein
the processor is configured to
further determine whether there is a sign of rupture of the membrane, and whether the membrane is high-performance based on an analysis result obtained.

3. The analysis device according to claim 1, wherein
the processor is configured to
detect presence of a target microorganism out of microorganisms including a virus in the permeate water, by the PCR analysis.

4. The analysis device according to claim 1, wherein
the processor is configured to
perform PCR analysis with respect to the permeate water over a certain period of time, and
determine whether a rupture has occurred in the period of time according to sensitivity characteristics of the PCR analysis and a result of the PCR analysis.

5. The analysis device according to claim 1, wherein
determine that a rupture in a size according to a size of a microorganism has occurred in the membrane, the microorganism detected by the PCR analysis performed by the analyzing unit.

6. The analysis device according to claim 1, wherein
the processor is configured to
perform PCR analysis with respect to permeate water collected from a UF membrane.

7. The analysis device according to claim 1, wherein
the processor is configured to
perform PCR analysis with respect to permeate water collected from an MF membrane.

8. The analysis device according to claim 1, wherein
the processor is configured to
perform PCR analysis with respect to permeate water that is concentrated after collection.

9. The analysis device according to claim 1, wherein
the processor is configured to
output information relating to a kind of microorganism detected, and information relating to a size of a rupture according to the kind of the microorganism.

10. An analysis method that is performed by a computer, the method comprising:
performing PCR analysis with respect to permeate water collected from a membrane that passes water therethrough; and
determining whether the membrane is ruptured based on an analysis result obtained by the analyzing process.

11. A non-transitory computer-readable recording medium having stored therein a program that causes a computer to execute a process comprising:
performing PCR analysis with respect to permeate water collected from a membrane that passes water therethrough; and
determining whether the membrane is ruptured based on an analysis result obtained by the analyzing step.

12. A water treatment system comprising:
a container; and
an analysis device, wherein
the container includes
a membrane module including a plurality of membranes that pass water therethrough;
a pipe to supply water to the membrane module;
a pipe to collect water from the membrane module; and
a pipe to connect to a device included in a system that treats sewage, and
the analysis device comprising:
a processor configured to:
perform PCR analysis with respect to permeate water collected from at least either one of the membranes;
determine whether the membrane is ruptured based on an analysis result obtained; and
output a determination result obtained.
